(19) United States
(12) Patent Application Publication
(10) Pub. No.: US 2026/0345928 A1
(43) Pub. Date: Nov. 5, 2026

(54) AN INHALER FOR POWDERED SUBSTANCES

(71) Applicant: Alfred VON SCHUCKMANN, Kevelaer (DE)

(72) Inventor: Alfred VON SCHUCKMANN, Kevelaer (DE)

(21) Appl. No.: 19/488,877

(22) PCT Filed: May 29, 2024
(86) PCT No.: PCT/EP2024/064783

§ 371 Date: Dec. 1, 2025

(30) Foreign Application Priority Data
Jun. 2, 2023 (DE) 10 2023 114 584.3

(51) Int. Cl. A61M 15/00

(52) U.S. Cl. CPC A61M 15/0041; A61M 15/0026; A61M 2202/0007; A61M 2202/064; A61M 2206/16

(57) ABSTRACT

An inhaler includes a housing with a base portion and a mouth piece, the base portion including a chamber that is adapted to receive a capsule containing powdered substances, the base portion further including a first capsule piercing means and a second capsule piercing means at opposite sides of the chamber adapted to pierce the capsule located within the chamber from opposite sides, wherein the inhaler further includes a first actuation means and a second actuation means slidably arranged at the base portion and capable of moving the first capsule piercing means and the second capsule piercing means relative to the chamber. In order to facilitate the usage, it is suggested that the first actuation means is gear coupled to the second actuation means such that a user operated pushing of the first capsule piercing means pulls the second capsule piercing means into the piercing position.

### FIELD OF TECHNOLOGY

The invention relates to an inhaler for powdered substances comprising a housing with a base portion and a mouth piece, the base portion comprising a chamber that is adapted to receive a capsule containing said powdered substances, the base portion further comprising a first capsule piercing means and a second capsule piercing means at opposite sides of the chamber adapted to pierce the capsule located within the chamber from opposite sides, wherein the chamber defines a piercing position for the capsule between the first capsule piercing means and the second capsule piercing means, the mouth piece comprising an inhalation passage connecting the chamber of the base portion to an outside of the housing, wherein the inhaler further comprises an air inlet in fluid communication with the inhalation passage, and user operated actuation means comprising a first actuation means and a second actuation means slidably arranged at the base portion and capable of moving the first capsule piercing means and the second capsule piercing means relative to the chamber in order to pierce the capsule.

### PRIOR ART

Inhalers according to the background of the invention are known in the state of the art. Such inhalers are used for inhaling the contents of a capsule, especially for medical uses. When using the inhaler, a user sucks air through the inhalation passage of the inhaler by sucking air at the mouth piece that is in fluid communication with the air inlet. Before inhaling the user operates two actuation elements, for example push-buttons, each moving a respective capsule piercing means to one of the opposite sides of the capsule. After piercing the capsule the substances are emitted and mixed with the air stream within the inhalation passage.

EP 1 270 034 B1 discloses an inhaler device with an inhaler body defining a recess for holding therein a capsule containing substances to be inhaled and a nosepiece communicating with the recess, wherein the inhaler comprises two opposite perforating means associated with the inhaler body and adapted to perforate the capsule to allow an outside airflow to be mixed with the contents of the capsule for inhalation through the nosepiece. Each perforating means comprises a perforation needle designed for transversely sliding against the biasing of a single urging coil spring encompassing said needle and operating between an abutment element, rigid with said inhaler body and a corresponding operating hollow push-button element. By pressing the push-button elements, the user forces the corresponding perforating needle into the respective side of the capsule.

According to the mentioned state of the art, a user needs to force two push-buttons simultaneously into the inhaler housing in order to perform an opening of the capsule. This may be uncomfortable for the user and may lead to an inhomogeneous mixture of substances with the airflow.

### SUMMARY OF THE INVENTION

Starting from the state of the art, the aim of the present invention is to provide an inhaler that can be used in a more comfortable and reproducible way.

According to a first idea, a possible solution is provided in the case of an inhaler, wherein the first actuation means is geared coupled to the second actuation means such that a user operated pushing of the first capsule piercing means pulls the second capsule piercing means into the piercing position. Accordingly, the user only has to operate the first actuation means in order to move both the first capsule piercing means and the second capsule piercing means towards each other. It is not necessary that the user actuates a first actuation means for the first capsule piercing means and a second actuation means for the second capsule piercing means. Instead, the first actuation means both actuates the first capsule piercing means and the second capsule piercing means at least indirectly. Especially, the first actuation means pulls the second capsule piercing means that is geared coupled to the first actuation means towards the first capsule piercing means and thereby to the chamber that holds a capsule in its piercing position. The movement of the first actuation means and the second actuation means is opposite to each other such that a pushing of the first actuation means leads to a pulling of the second actuation means or the other way around. Furthermore, a respective pushing of the first capsule piercing means leads to a pulling of the second capsule piercing means.

The first and second actuation means and the first and second capsule piercing means are arranged one behind the other and move towards each other in order to perform a piercing, when the first actuation means and the second actuation means move opposite to one another.

Thanks to the gear coupling between the first actuation means and the second actuation means the inhaler has to provide only one actuation element, for example an actuation element that belongs to the first actuation means. This actuation element has to protrude beyond the housing of the inhaler in order to be actuated by the user, for example by pushing the actuation element towards the housing, especially further into the housing. Therefore, the actuation means, namely the first actuation means or the second actuation means, respectively, is slidably arranged at the base portion. It may be possible alternatively that the user operates the actuation means by pulling the first actuation means out of the housing of the inhaler, but that may not be a preferable embodiment.

In this way, the actuation means comprises only one actuation element and coupling means connected to the only one actuation element and to each of the first capsule piercing means and the second capsule piercing means, wherein by actuation of the actuation means the first capsule piercing means and second capsule piercing means are moved towards each other and towards opposite ends of the capsule in order to pierce the capsule from opposite sides simultaneously.

The first actuation means is coupled to the second actuation means by coupling means. The coupling means transfers a movement of the first actuation means to the second actuation means in order to provide that the user only has to operate the first actuation means. In this sense, both the first and second actuation means are moved in response to a user induced operation of the actuation means. The second actuation means is thereby actuated indirectly as a follow-up of the movement of the first actuation means.

The coupling means of the inhaler may provide different kinds of gear coupling. For example, the coupling means may comprise a seesaw mechanism, a gear wheel, a gear rack or others.

It is proposed, that the coupling means is located below the chamber. Especially, the coupling means is located preferably outside the chamber such that the coupling means does not interfere with a capsule that is located in a piercing position within the chamber, especially between the first capsule piercing means and the second capsule piercing means.

According to one possible embodiment the coupling means comprises a seesaw mechanism with a crossbar pivoted about a rotation axis relative to the base portion, the rotation axis dividing the crossbar into two opposite crossbar ends, wherein a first crossbar end is connected to the first capsule piercing means and wherein a second crossbar end is connected to the second capsule piercing means.

The inhaler needs only one actuation element to be actuated by a user for piercing the capsule. However, there are still two capsule piercing means with holding means for separate piercing needles, namely the first capsule piercing means and the second capsule piercing means. Due to the connection of only one actuating element with the coupling means, the user can move both capsule piercing means towards opposite sides of the capsule by only one single operation. Especially, the actuation element is connected to only one crossbar end of the crossbar of the seesaw mechanism. As a follow-up of a pushing motion of the first capsule piercing means (and the actuation element) the second capsule piercing means is pulled towards the piercing position of the capsule due to a movement of the other crossbar end around the rotation axis of the crossbar.

Only one of the capsule piercing means is directly actuatable by a user by operating the actuation means. The other capsule piercing means is internally coupled to the first capsule piercing means by the seesaw mechanism. Thereby, the inhaler housing provides only one single actuation element for the user, which however actuates both the first capsule piercing means and the second capsule piercing means, when the user actuates the coupling means by for example pushing the actuation element. In contrast to the state of the art, the user does not have to push two actuation elements simultaneously in order to pierce the capsule from two opposite sides, neither does the user have to use two hands in order to slide a closure cap about two opposite sides of the inhaler housing which requires the user to retain the housing by a first hand and to push the slidable cover cap by a second hand.

As the outside of the inhaler shows only one single actuation element, the user receives a clear guidance where to push in order to pierce the capsule. Alternatively to a seesaw mechanism other transfer mechanisms for transferring the motion of the first actuation means to the second actuation means can be provided, for example mechanisms comprising gear wheels or gear racks.

With regard to a plane of the inhaler, in which the capsule lies parallel with regard to its longitudinal axis, the actuation means is also oriented such that the sliding motion of the actuation element is oriented parallel or at least essentially parallel to this plane and thus parallel or at least essentially parallel to the longitudinal extension of the capsule, respectively. The moving direction and also piercing direction of the first capsule piercing means and the second capsule piercing means is therefore also parallel or at least essentially parallel to this plane. In consequence, the capsule is pierced from opposite sides. For example, the rotation axis of a crossbar of a seesaw mechanism can be oriented perpendicularly to the moving direction of the actuation element and also perpendicular to the longitudinal extension of the capsule.

Regarding a gear coupling having a seesaw mechanism, the actuation element is connected to only one crossbar end of the crossbar, whereby a linear sliding motion of the actuation element leads to a rotation of the crossbar around the rotation axis and thereby also to a rotation movement of the other, second crossbar end of the crossbar. With regard to an embodiment including the first capsule piercing means connected to the first crossbar end and the second capsule piercing means connected to the second crossbar end as well as the first capsule piercing means and the second capsule piercing means facing the crossbar from different sides in a plane perpendicular to the rotation axis of the crossbar, this leads to a counter movement of the first capsule piercing means and the second capsule piercing means and thereby piercing the capsule from opposite sides.

According to another, alternative embodiment, the rotation axis of the crossbar could be oriented parallel to a plane in which the capsule piercing means are moved and in which the longitudinal axis of the capsule lies. The assembly of the seesaw mechanism and especially its elements, like the crossbar, is particularly related to the shape of the inhaler housing, especially the shape and size of the base portion, in which the seesaw mechanism is located.

With regard to for example an embodiment, in which the base portion is disc-shaped the rotation axis of the crossbar could be oriented preferably perpendicular to the main flat extension of the disc-shape. The shape of the base portion is however not restricted to a specified shape. Instead, the base portion can be formed for example circular, oval, square, polygonal or following a free form.

According to an embodiment, the first crossbar end is jointly connected to a first intermediate arm connected to the first capsule piercing means, and the second crossbar end is jointly connected to a second intermediate arm connected to the second capsule piercing means. Although the first crossbar end and the second crossbar end, respectively, could be connected directly to the corresponding capsule piercing means, a preferred embodiment proposes connecting the crossbar end and the respective capsule piercing means by an intermediate arm that is pivotable connected to a corresponding crossbar end. According to a preferred embodiment, the intermediate arm is also pivotable mounted to the respective capsule piercing means in order to transform a linear motion of the capsule piercing means or the actuation element, respectively, to a circular movement of the crossbar.

The connection between the crossbar end and the intermediate arm and/or between the intermediate arm and the capsule piercing means can be implemented by a screw connection, a rivet connection, a one-piece connection, an elastic joint or other connection types. Especially the elastic joint can be a one-piece connection.

In the case of other types of gear coupling, the mechanical principle of opposite push-pull-motions applies accordingly.

An essential feature of the suggested embodiments is the counter movement of the first capsule piercing means and the second capsule piercing means. If actuated the actuation element preferably moves in a direction parallel to the piercing direction of the first capsule piercing means or the second capsule piercing means, respectively.

It is suggested, that only the first actuation means of the first and second actuation means comprises an actuation element that is operable by a user. This actuatable element preferably protrudes beyond a contour of the housing, whereas the second actuation means is located invisible inside of the housing. Thereby, the user receives a clear indication where to push the inhaler in order to start an inhalation process.

With regard to the type of actuation element, it is suggested that the actuation element of the actuation means is a push-button element. A push-button element provides a linear movement direction and can be preferably oriented parallel to the longitudinal extension of the capsule to be pierced and thus the movement direction of the capsule piercing means. Further, it is always clear to a user of the inhaler how to actuate a push-button element, especially in a direction perpendicular to an outer contour of the housing of the inhaler.

According to a preferred embodiment, the first capsule piercing means is directly mounted to or fabricated as one piece with a flipside of the actuation element. Thereby, a movement of the actuation element directly results in a movement of the first capsule piercing means. The first capsule piercing means is located on the flipside of the actuation element. The capsule piercing means on the flipside of the actuation element is moved in the actuation direction towards an end side of the capsule to be pierced.

The first capsule piercing means can be mounted on the actuation element by different connection technologies, for example by screwing, overmolding or gluing. Further, the actuation element and the first capsule piercing means can be fabricated as one piece, wherein a front side is pushed by a user of the inhaler and wherein the flipside has the capsule piercing means facing towards the capsule end.

At least the first and/or second actuation means slides preferably against a reset force of a spring element. According to one embodiment the spring element can be a coil spring encompassing a part of the first capsule piercing means and/or the actuation element, respectively. Although not necessary, also both capsule piercing means can slide against a biasing of a spring element, provided for at each of the capsule piercing means. Further preferably, only at one of the capsule piercing means a spring is provided for. The spring element ensures that the actuation element and the capsule piercing means are restored to an initial default position in which the actuation element preferably protrudes over a contour of the inhaler housing preferably by a maximum. Further, the spring element ensures that the capsule piercing means is removed from the capsule after the capsule has been pierced in order to ensure that the substances can be emitted from the capsule.

Furthermore it is suggested according to one possible embodiment that the chamber of the base portion comprises a capsule rotation area and a recess for placing the capsule in the piercing position next to the corresponding capsule piercing means, wherein the capsule is movable out of the recess into the capsule rotation area by an air stream sucked by a user, and wherein the capsule is rotated by the air stream about a capsule axis that is oriented perpendicular to a longitudinal axis of the capsule thereby emitting the substances out of the capsule. After the capsule has been pierced at the opposite two end faces, the capsule is moved by an air stream sucked in by the user from the recess, where the capsule has been pierced, to the capsule rotation area. In the capsule rotation area, the capsule is turning about its rotation axis running perpendicular to a longitudinal axis of the capsule. The rotational movement is caused by the air stream which enters the inhaler through the air inlet which is in fluid communication with the inhalation passage. Preferably, the air stream propagates within a circular rotation area, thereby inducing a rotational movement of the capsule. The substances stored in the capsule are ejected due to the centrifugal force acting on the capsule and thereby also acting on the substances. This functionality is independent of an internal airway between the pierced end surfaces of the capsule. The substances mix with the air in the inhalation passage and pass through the mouth piece to the user.

Furthermore, it is proposed that the base portion comprises a capsule with two separate compartments for the powdered substances, wherein the compartments succeed each other with regard to a longitudinal axis of the capsule and are separated by a separating wall. According to this embodiment, the capsule may store two different substances, wherein a first substance is stored in a first compartment and a second substance is stored in a second compartment of the capsule. After each compartment has been pierced by a respective capsule piercing means, the substances of both compartments can be ejected due to the centrifugal force acting on the substances in both compartments. Further, as previously described, by applying suction on the mouth piece, an airflow is generated, which, coming from the air inlet, enters the chamber, thereby mixing with the capsule content.

According to a further preferred embodiment, the capsule has a cylindrical shape with two opposite dome-shaped front faces. Due to the especially inside dome-shaped front faces, the substances stored in the capsule are led to the capsule openings at the front faces, where the respective front face has been pierced by the capsule piercing means. As in contrast to flat front faces of a capsule, the dome-shaped front faces prevent sticking of a part of the substances on for example an edge of the front face.

Especially, the first capsule piercing means is adapted to pierce with regard to the separating wall of the capsule, a radially outward pointing front face of a first compartment of the capsule, and the second capsule piercing means is adapted to pierce, with regard to the separating wall, a radially outward pointing front face of a second compartment of the capsule. Therefore, the inhaler is especially build to spend a mix of different substances within an inhalation air stream to be sucked in by a user. The inhaler is capable preferably for piercing two opposite ends of a capsule that has two separate compartments separated by a separating wall simultaneously, that means especially at the same time.

Alternatively, the piercing of the capsule by the first capsule piercing means and the piercing of the capsule by the second piercing means could however have a time delay that could be caused for example by different lengths of two opposite crossbar ends of a seesaw mechanism and/or by different lengths of a first intermediate arm and a second intermediate arm that connect a respective crossbar end to either the first capsule piercing means or the second capsule piercing means. By applying different lever length, a first front face of the capsule could be contacted by the first capsule piercing means before a second front face of the capsule is contacted by the second capsule piercing means.

### BRIEF DESCRIPTION OF THE DRAWINGS

Further characteristics and advantages of the inhaler will become more apparent hereinafter from the following detailed disclosure of a preferred, though not exclusive, embodiment of the invention, which is illustrated, by way of an indicative, but not limitative example, in the accompanying drawings, where

FIG. 1 shows a perspective view of an inhaler according to an embodiment,

FIG. 2 shows a side view of the inhaler according to FIG. 1,

FIG. 3 shows a bottom side view of the inhaler according to the direction III in FIG. 1,

FIG. 4 shows a perspective view of the inhaler in an open position of the inhaler housing (capsule loading position),

FIG. 5 shows an exploded perspective view of the inhaler,

FIG. 6 shows a cross sectional view of the inhaler in a start position,

FIG. 7 shows a cross sectional view of the inhaler according to the direction VII in FIG. 6,

FIG. 8 shows a cross sectional view of the inhaler device with a capsule arranged in a chamber of a base portion (piercing position),

FIG. 9 shows a cross sectional view of the inhaler according to the direction IX in FIG. 8,

FIG. 10 shows a similar view to FIG. 8, but illustrating a capsule piercing operation,

FIG. 11 shows a cross sectional view of the inhaler according to the direction XI in FIG. 10,

FIG. 12 shows an enlarged area according to the area XII of the inhaler of FIG. 10,

FIG. 13 shows the capsule entering a capsule rotation area of the chamber.

### DESCRIPTION OF THE PREFERRED EMBODIMENTS

Shown is an exemplary embodiment of an inhaler 1 with a housing 3 comprising a base portion 4 and a mouth piece 5. The inhaler 1 is designed for emitting a medical substance 2 that could be inhaled by a user of the inhaler 1.

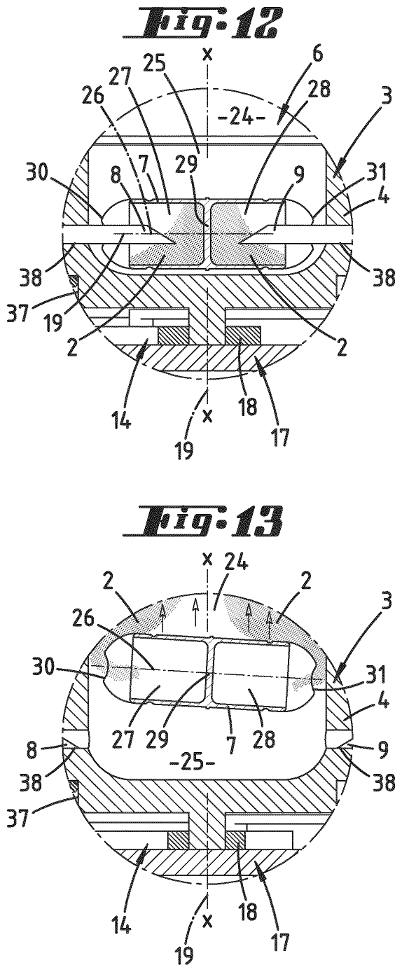

Furthermore, according to a possible embodiment, the inhaler 1 is capable of mixing for example two different pharmaceutical substances 2 that are stored in two separate compartments 27, 28 of a capsule 7 (see especially FIGS. 12 and 13).

The base portion 4 of the housing 3 comprises a bottom 33 and a chamber 6 with a recess 25 for holding a capsule 7 in a piercing position and a capsule rotation area 24. The capsule rotation area 24 is arranged above the recess 25, that means with regard to the bottom 33 of the base portion 4 on the opposite side of the recess 25. The recess 25 has a longitudinal shape in order to store a capsule 7 having an elongated, especially cylindrical shape.

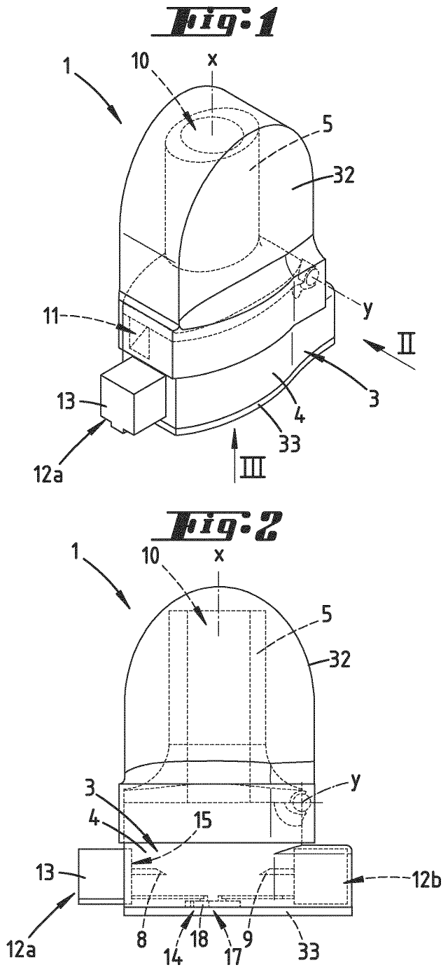

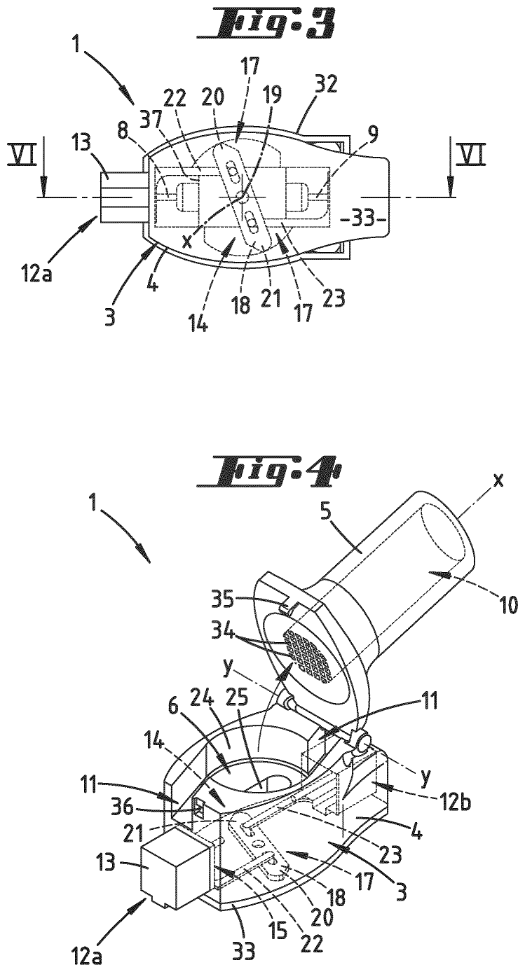

As according to for example FIGS. 1 and 2, the inhaler 1 comprises a cap 32 that covers the mouth piece 5 of the inhaler 1. The mouth piece 5 and the cap 32 have an elongated shape along an inhaler axis x. The cap 32 can be removed from the mouth piece 5 and the base portion 4 of the housing 3.

The mouth piece 5 provides an inhalation passage 10 that runs from the chamber 6 of the base portion 4 to an outside of the mouth piece 5, where the user may suck air in order to inhale the medical substance 2. A protective grid 34 is located between the inhalation passage 10 and the chamber 6 in order to prevent larger particles, especially the capsule 7, from entering the inhalation passage 10 and being inhaled by a user. This ensures safety of the user.

Furthermore, the chamber 6, here for example the capsule rotation area 24, has two air inlets 11, through which air can pass into the inhaler 1, when the user sucks air at the mouth piece 5.

Although the inhaler 1 comprises according to this embodiment a mouth piece 5, the inhaler 1 could alternatively provide a nosepiece to enable a nasal application. The described functionality of the inhaler 1 is however the same.

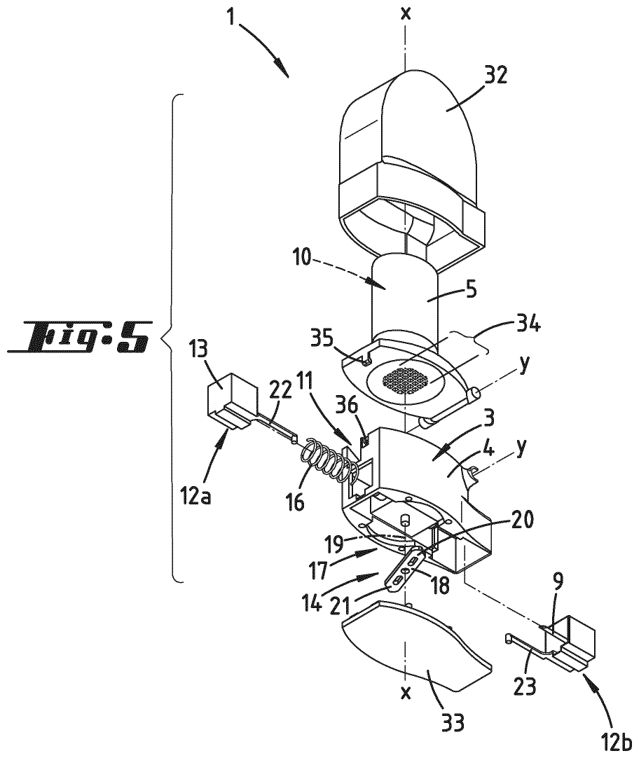

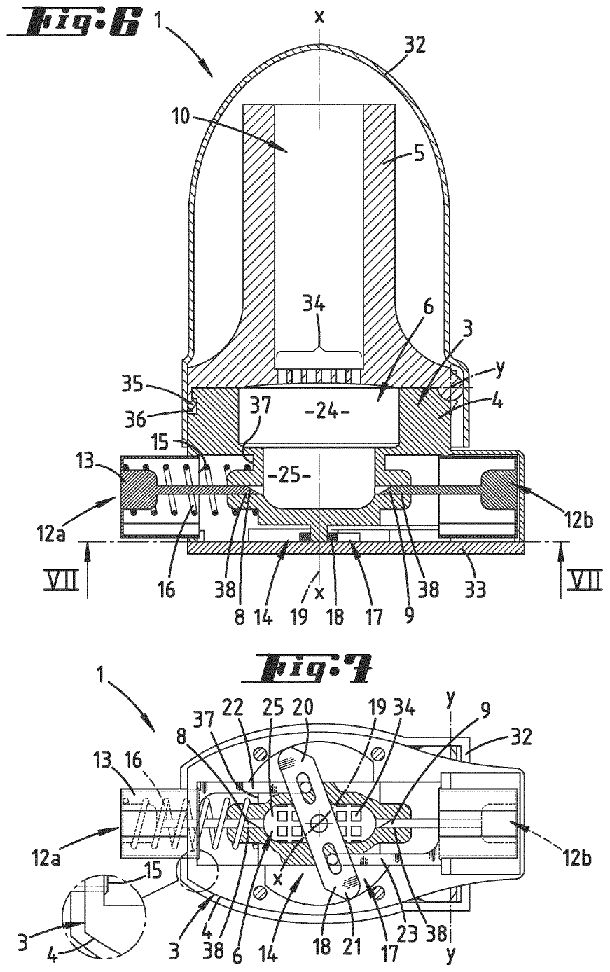

As shown for example in FIG. 4, the mouth piece 5 can be tilted relative to the base portion 4 about a pivot axis y in order to open the chamber 6 for inserting a capsule 7 into the chamber 6, namely especially into the recess 25. The recess 25 has preferably a shape that fits to the shape of the capsule 7 to be inserted. The capsule rotation area 24, which is located above the recess 25, has on the contrary a circular shape in order to enable a rotation of the capsule 7 within the capsule rotation area 24 after the capsule 7 has been sucked by an air stream from the recess 25 into the capsule rotation area 24. Due to the air stream that is sucked into the chamber 6 through the air inlets 11, the capsule 7 is on the one hand lifted up from the recess 25 into the capsule rotation are 24, and on the other hand rotated about a capsule rotation axis (not shown) that is oriented perpendicular to a longitudinal axis 26 of the capsule 7. The capsule rotation axis coincides here for example essentially with the inhaler axis x. However, the orientation of the rotation axis may vary due to a tumbling motion of the capsule 7.

The tiltable mouth piece 5 can be secured to the base portion 4 by a hook 35 of the mouth piece 5. The hook 35 can be engaged into a corresponding groove 36 of the base portion 4, or the other way around. Alternative to a hook-groove-connection other fastening functionalities could be provided.

In order to release the medical substances 2 out of the capsule 7, the inhaler 1 comprises a first actuation means 12a with an actuation element 13 that protrudes about the housing 3 of the inhaler 1 and a second actuation means 12b. The actuation element 13 is a single element, here designed as a push-button. On a flipside 15 of the actuation element 13 is provided a first capsule piercing means 8 in the form of a needle for piercing an opening into a front face 30 of the capsule 7.

The actuation element 13 is connected to a coupling means 14 which gear couples the first actuation means 12a to the second actuation means 12b such that a user operated pushing of the first capsule piercing means 8 pulls the second capsule piercing means 9 into the piercing position.

Here, the coupling means 14 comprises as one embodiment of a gear coupling a seesaw mechanism 17 that transfers the sliding motion of the actuation element 13 and of the first capsule piercing means 8 to a second capsule piercing means 9 that is located on an opposite side of the coupling means 14.

The seesaw mechanism 17 has a central crossbar 18 that is rotatable about a rotation axis 19 fixed to the base portion 4 of the inhaler. The rotation axis 19 may for example coincide with the inhaler axis x. The crossbar 18 comprises a first crossbar end 20 connected to the first capsule piercing means 8 of the actuation element 13 and a second crossbar end 21 connected to the second capsule piercing means 9. The second capsule piercing means 9 does not protrude over a contour of the housing 3. Although the first crossbar end 20 and the second crossbar end 21, respectively, could be directly connected to the corresponding capsule piercing means 8, 9, here intermediate arms 22, 23 are provided.

The first intermediate arm 22 connects the flipside 15 of the actuation element 13 to the first crossbar end 20 of the crossbar 18. The second intermediate arm 23 connects the second crossbar end 21 to the second capsule piercing means 9. By actuation, that means sliding of the actuation element 13 relative to the base portion 4 of the inhaler 1, the first intermediate arm 22 pushes the first crossbar end 20 of the crossbar 18 towards the second capsule piercing means 9. Thereby, the crossbar 18 rotates about the rotation axis 19, which leads to an opposite swing of the second crossbar end 21, which in consequence forces the second capsule piercing means 9 to propagate further in a direction towards the first capsule piercing means 8.

The first capsule piercing means 8 and the second capsule piercing means 9 drive towards each other as a result of actuating only the single actuation element 13 by a user of the inhaler 1. The linear sliding of the actuation element 13 leads to a simultaneous movement of both capsule piercing means 8, 9 toward opposite front faces 30, 31 of the capsule 7.

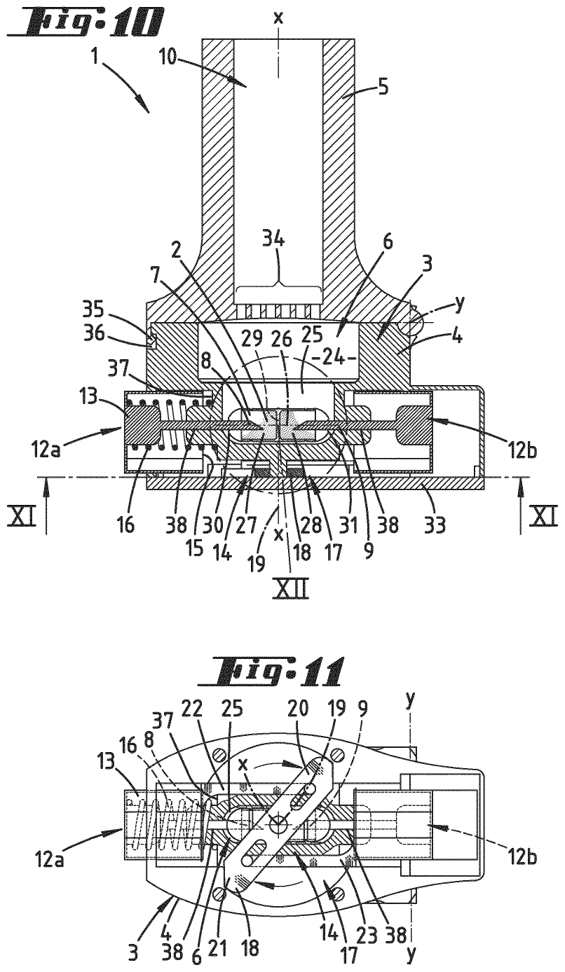

Here, as can be seen for example in FIGS. 9 to 13, the capsule 7 has two separate compartments 27, 28, which are separated by a separating wall 29. Here, the compartments 27, 28 are identically formed and have an identical volume. However, the compartments 27, 28 could also be different from each other. Each compartment 27, 28 can store a different substance 2, although the substances 2 of the compartments 27, 28 could also be the same.

Further, a spring element 16 is assigned to the actuation element 13 and the connected first capsule piercing means 8, respectively (as shown for example in FIG. 5). The spring element 16 has a reacting force that acts against the pushing force of the user on the actuation element 13. Here, the spring element 16 is a coil spring that surrounds the first capsule piercing means 8 on the flipside 15 of the actuation element 13. However, also other types of spring elements 16 could be applied. The spring element 16 operates between an abutment element 37 of the base portion 4 of the inhaler 1 and an inner surface of the actuation element 13 defining a cavity in which the first capsule piercing means 8 is located.

After the user releases the actuation element 13, the spring element 16 elongates again and pushes the actuation element 13 out of the housing 3 of the inhaler 1. After that, the inhaler 1 and its actuation element 13, respectively, are ready for a new inhalation step.

Regarding the longitudinal extension of the recess 25, the base portion 4 of the inhaler 1 has guideways 38 for guiding the corresponding capsule piercing means 8, 9, namely the respective needle. Each piercing means 8, 9 passes through the corresponding guideway 38 and slides into the volume of the recess 25 defining the piercing position of the capsule 7, if the user activates the gear coupling by pushing the actuation element 13 on the outside of the housing 3. If the user releases the actuation element 13, the piercing means 8, 9 pass through the guideways 38 in opposite direction.

As mentioned before, the capsule rotation area 24 has two air inlets 11, which enter the capsule rotation area 24 on opposite sides of the circumference. The orientation and size of each air inlet 11 is chosen such that the air passing into the chamber 6 from the outside is preferably tangential with regard to the circular capsule rotation area 24, in order to induce a rotating air stream along the inner circumference of the capsule rotation area 24. Besides this, there is provided an open connection between the chamber 6, especially the capsule rotation area 24, the inhalation passage 10 and the air inlets 11, thereby also providing a connection to the outside of the inhaler 1.

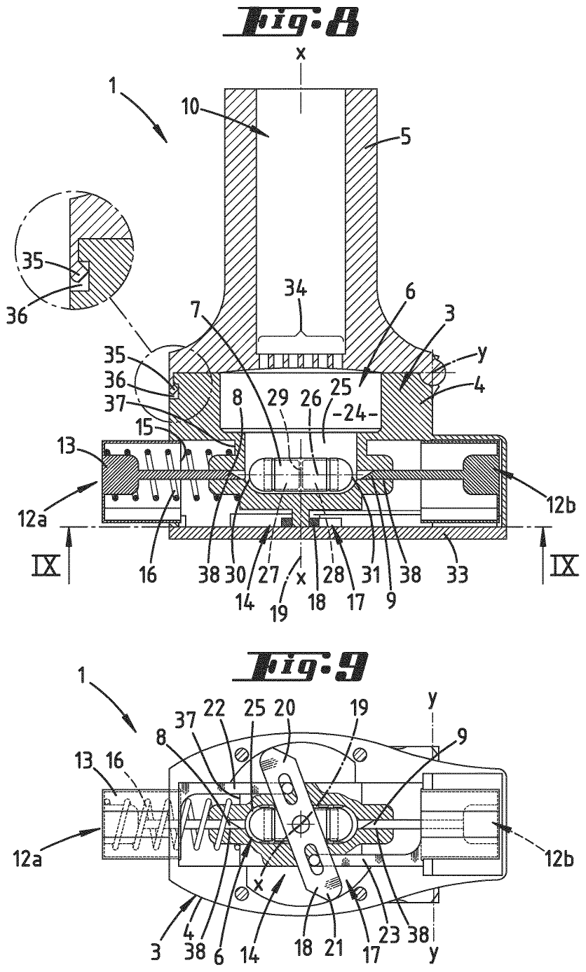

With regard to the capsule 7 itself, it is pointed to especially FIGS. 8 to 13. The capsule 7 has a cylindrical base shape with two dome-shaped front faces 30, 31 and two separate compartments 27, 28, which are separated by the separating wall 29. However, the capsule 7 could alternatively provide only one single compartment 27, 28. It is not necessary that the compartments 27, 28 have a direct fluid connection to each other. The separating wall 29 instead fully separates the two compartments 27, 28 from each other.

Further, the capsule 7 could be fabricated out of mainly three parts, for example a central part comprising the separating wall 29 and two dome-shaped front parts that fluidly seal and form one or two compartments 27, 28 of the capsule 7. For example, especially the dome-shaped front faces 30, 31 of the capsule 7 could be made from gelatine.

In a preferred embodiment, the first compartment 27 of the capsule 7 comprises a different substance 2 than the second compartment 28 of the capsule 7. Both substances 2 can be inhaled simultaneously by a user during an inhalation process, wherein the two substances 2 are only mixed in especially the moment when the user sucks air at the mouth piece 5.

At first, before starting an inhalation, the user could open the housing 3 of the inhaler 1 by distancing the mouth piece 5 from the base portion 4. Therefore, the user could swivel the mouth piece 5 about the pivot axis y. Then, the user inserts the capsule 7 into the recess 25 of the chamber 6 (as for example according to FIG. 8). Afterwards the user closes the housing 3 by swivelling the mouth piece 5 back about the pivot axis y until a contact to the base portion 4 is given.

Then, the user pushes the actuation element 13 against the restoring force of the spring element 16. The actuation element 13 activates the coupling means 14 by pushing the crossbar 18 of the seesaw mechanism 17 at the first crossbar end 20. This results into a pivoting motion of the crossbar 18 about the rotation axis 19, so that also the second crossbar end 21 is swivelled about the rotation axis 19, thereby pulling the second capsule piercing means 9 in the direction of the recess 25 and the capsule 7 therein. The way of movement of the first capsule piercing means 8 and the second capsule piercing means 9 is preferably chosen such that the separating wall 29 is not contacted by the capsule piercing means 8, 9, so that the compartments 27, 28 remain separated from each other.

As soon as the user releases the actuation element 13, the seesaw mechanism 17 returns into a starting position due to the restoring force of the spring element 16. Then, the inhaler 1 is ready for inhalation by the user. Both compartments 27, 28 are pierced at their front faces 30, 31, so that the substances 2 can be emitted from the capsule 7.

When the user closes his or her lips around the end portion of the mouth piece 5, he or she can suck air through the inhalation passage 10 of the mouth piece 5, the chamber 6 and the air inlets 11. Thereby a suction air stream is generated within the inhaler 1. This air stream forces the (pierced) capsule 7 to move from the recess 25 into the capsule rotation area 24. Due to the position of the air inlets 11, the capsule 7 starts to rotate within the capsule rotation area 24 around a rotation axis that is perpendicular to the longitudinal axis 26 of the capsule 7. The protective grid 34 however prevents the capsule 7 from entering the inhalation passage 10. Preferably, the rotation axis of the capsule 7 is identical to the inhaler axis x. However, during the rotational movement of the capsule 7, a tumbling motion of the capsule 7 could also occur.

Due to the rotational movement and occasionally the tumbling motion of the capsule 7, the substances 2 leave the respective compartment 27, 28 of the capsule 7 and mix. The dome-shaped front faces 30, 31 prevent the substances 2 from sticking and lead the substances 2 to the holes of the front faces 30, 31, where the capsule piercing means 8, 9 have pierced the capsule 7. Due to the rotational movement of the capsule 7, the substances 2 are emitted into the same rotational plane, which enhances the mixing process. A centrifugal force acting on the front faces 30, 31 of the capsule 7 enhances the depletion of the capsule 7.

After completing the inhalation process, the user could furthermore open the housing 3 of the inhaler 1 and extract the capsule 7 from the chamber 6 of the inhaler 1.

It is to be understood that the gear coupling that couples the first actuation means to the second actuation means can comprise other coupling means instead of a seesaw mechanism. The seesaw mechanism is here only provided as one possible embodiment of a gear coupling out of multiple other possible embodiments.

The above statements serve to explain the solutions covered by the application as a whole, which each also independently advance the prior art at least by the following feature combinations, wherein two, several or all of these feature combinations can also be combined, specifically:

An inhaler 1 characterized in that the first actuation means 12a is gear coupled to the second actuation means 12b such that a user operated pushing of the first capsule piercing means 8 pulls the second capsule piercing means 9 into the piercing position.

An inhaler 1 characterized in that the first actuation means 12a is coupled to the second actuation means 12b by coupling means 14.

An inhaler 1 characterized in that the coupling means 14 is located below the chamber 6.

An inhaler 1 characterized in that the coupling means 14 comprises a seesaw mechanism 17 with a crossbar 18 pivoted about a rotation axis 19 relative to the base portion 4, the rotation axis 19 dividing the crossbar 18 into two opposite crossbar ends 20, 21, wherein a first crossbar end 20 is connected to the first capsule piercing means 8 and wherein a second crossbar end 21 is connected to the second capsule piercing means 9.

An inhaler 1 characterized in that the first crossbar end 20 is jointly connected to a first intermediate arm 22 connected to the first capsule piercing means 8, and wherein the second crossbar end 21 is jointly connected to a second intermediate arm 23 connected to the second capsule piercing means 9.

An inhaler 1 characterized in that only the first actuation means 12a of the first and second actuation means 12a, 12b comprises an actuation element 13 that is operable by a user.

An inhaler 1 characterized in that the actuation element 13 is a pushbutton element.

An inhaler 1 characterized in that the first capsule piercing means 8 is directly mounted to or fabricated as one-piece with a flipside 15 of the actuation element 13.

An inhaler 1 characterized in that at least the first actuation means 12a slides against a reset force of a spring element 16.

An inhaler 1 characterized in that the chamber 6 of the base portion 4 comprises a capsule rotation area 24 and a recess 25 for placing the capsule 7 in the piercing position next to the corresponding capsule piercing means 8, 9, wherein the capsule 7 is movable out of the recess 25 into the capsule rotation area 24 by an air stream sucked by a user, and wherein the capsule 7 is rotated by the air stream about a capsule axis that is oriented perpendicular to a longitudinal axis 26 of the capsule 7 thereby emitting the substances 2 out of the capsule 7.

An inhaler 1 characterized in that the chamber 6 of the base portion 4 comprises a capsule 7 with two separate compartments 27, 28 for the powdered substances 2, wherein the compartments 27, 28 succeed each other with regard to a longitudinal axis 26 of the capsule 7 and are separated by a separating wall 29.

An inhaler 1 characterized in that the capsule 7 has a cylindrical shape with two opposite dome-shaped front faces 30, 31.

An inhaler 1 characterized in that the first capsule piercing means 8 is adapted to pierce, with regard to the separating wall 29, a radially outward pointing front face 30 of a first compartment 27 of the capsule 7, and wherein the second capsule piercing means 9 is adapted to pierce, with regard to the separating wall 29, a radially outward pointing front face 31 of a second compartment 28 of the capsule 7.

All disclosed features (whether taken separately or in combination with each other) are essential to the invention. The disclosure of the application hereby also incorporates the disclosure content of the accompanying/attached priority documents (copy of the prior application) in its entirety, also for the purpose of including features of these documents in claims of the present application. Even without the features of a referenced claim, the subclaims characterize standalone inventive further developments of prior art with their features, in particular so as to submit partial applications based upon these claims. The solution indicated in each claim can additionally have one or several of the features indicated in the above description, in particular those provided with reference numbers and/or indicated on the reference list. The described solution also relates to design forms in which individual features specified in the above description are not realized, in particular if they are recognizably superfluous with regard to the respective intended use, or can be replaced by other technically equivalent means.

Reference List

| | |
|---|---|
| 1 | Inhaler |
| 2 | Substance |
| 3 | Housing |
| 4 | Base portion |
| 5 | Mouth piece |
| 6 | Chamber |
| 7 | Capsule |
| 8 | First capsule piercing means |
| 9 | Second capsule piercing means |
| 10 | Inhalation passage |
| 11 | Air inlet |
| 12a | First actuation means |
| 12b | Second actuation means |
| 13 | Actuation element |
| 14 | Coupling means |
| 15 | Flipside |
| 16 | Spring element |
| 17 | Seesaw mechanism |
| 18 | Crossbar |
| 19 | Rotation axis |
| 20 | First crossbar end |
| 21 | Second crossbar end |
| 22 | First intermediate arm |
| 23 | Second intermediate arm |
| 24 | Capsule rotation area |
| 25 | Recess |
| 26 | Longitudinal axis |
| 27 | Compartment |
| 28 | Compartment |
| 29 | Separating wall |
| 30 | Front face |
| 31 | Front face |
| 32 | Cap |
| 33 | Bottom |
| 34 | Protective grid |
| 35 | Hook |
| 36 | Groove |
| 37 | Abutment element |
| 38 | Guideway |
| x | Inhaler axis |
| y | Pivot axis |

1: An inhaler (1) for powdered substances (2) comprising a housing (3) with a base portion (4) and a mouth piece (5), the base portion (4) comprising a chamber (6) that is adapted to receive a capsule (7) containing said powdered substances (2), the base portion (4) further comprising a first capsule piercing means (8) and a second capsule piercing means (9) at opposite sides of the chamber (6) adapted to pierce the capsule (7) located within the chamber (6) from opposite sides, wherein the chamber (6) defines a piercing position for the capsule (7) between the first capsule piercing means (8) and the second capsule piercing means (9), the mouth piece (5) comprising an inhalation passage (10) connecting the chamber (6) of the base portion (4) to an outside of the housing (3), wherein the inhaler (1) further comprises an air inlet (11) in fluid communication with the inhalation passage (10), and user operated actuation means (12a, 12b) comprising a first actuation means (12a) and a second actuation means (12b) slidably arranged at the base portion (4) and capable of moving the first capsule piercing means (8) and the second capsule piercing means (9) relative to the chamber (6) in order to pierce the capsule (7), and wherein the first actuation means (12a) is gear coupled to the second actuation means (12b) such that a user operated pushing of the first capsule piercing means (8) pulls the second capsule piercing means (9) into the piercing position.

2: The inhaler (1) according to claim 1, wherein the first actuation means (12a) is coupled to the second actuation means (12b) by coupling means (14).

3: The inhaler (1) according to claim 2, wherein the coupling means (14) is located below the chamber (6).

4: The inhaler (1) according to claim 2, wherein the coupling means (14) comprises a seesaw mechanism (17) with a crossbar (18) pivoted about a rotation axis (19) relative to the base portion (4), the rotation axis (19) dividing the crossbar (18) into two opposite crossbar ends (20, 21), wherein a first crossbar end (20) is connected to the first capsule piercing means (8) and wherein a second crossbar end (21) is connected to the second capsule piercing means (9).

5: The inhaler (1) according to claim 4, wherein the first crossbar end (20) is jointly connected to a first intermediate arm (22) connected to the first capsule piercing means (8), and wherein the second crossbar end (21) is jointly connected to a second intermediate arm (23) connected to the second capsule piercing means (9).

6: The inhaler (1) according to claim 1, wherein only the first actuation means (12a) of the first and second actuation means (12a, 12b) comprises an actuation element (13) that is operable by a user.

7: The inhaler (1) according to claim 6, wherein the actuation element (13) is a push-button element.

8: The inhaler (1) according to claim 6, wherein the first capsule piercing means (8) is directly mounted to or fabricated as one-piece with a flipside (15) of the actuation element (13).

9: The inhaler (1) according to claim 1, wherein at least the first actuation means (12a) slides against a reset force of a spring element (16).

10: The inhaler (1) according to claim 1, wherein the chamber (6) of the base portion (4) comprises a capsule rotation area (24) and a recess (25) for placing the capsule (7) in the piercing position next to the corresponding capsule piercing means (8, 9), wherein the capsule (7) is movable out of the recess (25) into the capsule rotation area (24) by an air stream sucked by a user, and wherein the capsule (7) is rotated by the air stream about a capsule axis that is oriented perpendicular to a longitudinal axis (26) of the capsule (7) thereby emitting the substances (2) out of the capsule (7).

11: The inhaler (1) according to claim 1, wherein the chamber (6) of the base portion (4) comprises a capsule (7) with two separate compartments (27, 28) for the powdered substances (2), wherein the compartments (27, 28) succeed each other with regard to a longitudinal axis (26) of the capsule (7) and are separated by a separating wall (29).

12: The inhaler (1) according to claim 11, wherein the capsule (7) has a cylindrical shape with two opposite dome-shaped front faces (30, 31).

13: The inhaler (1) according to claim 11, wherein the first capsule piercing means (8) is adapted to pierce, with regard to the separating wall (29), a radially outward pointing front face (30) of a first compartment (27) of the capsule (7), and wherein the second capsule piercing means (9) is adapted to pierce, with regard to the separating wall (29), a radially outward pointing front face (31) of a second compartment (28) of the capsule (7).
